SEQUENCE LISTING (30 sequences)

SEQ ID NO: 1 - DNA, 6234 bp, Human
atgacttcctcgctgcagcggccctggcgggtgccctggctaccatggactactgaaggagcgacgtcgccgggaccgcccacgggaccgatggtacctgcatcctgctggtcagcactgcggctgcttcgcagaatcaagaacggctatgtaggacgaccagtcgtgacgccgacgaagcgtcttagttcttgccgatagtgcgtttaaagatccgtatcagcaagaccttgggataggtgagagtagacacgcaaatttctaggcatagtcgttctggaaccctatccactctcatctatctctcatgaaaatgggacaatattatgctcgaaaggtagcacctgctatagagagtacttttaccctgttataatacgagctttccatcgtggacgattggcctttgggagaaatcaaaaggggacataaatcttgtaaaacaaggataccggaaaccctctttagttttcccctgtatttagaacattttgttcctagttggtctcacattggagatccccaagagtgtcactatgaagaatgtgtacaaccagagtgtaacctctaggggttctcacagtgatacttcttacacatgtaactaccactcctccctcaattcagaatggaacataccgtttctgctgcattgatggtgaggagggagttaagtcttaccttgtatggcaaagacgacttgtagcacagatttatgtaatgtcaactttactgagaattttccacctcaacatcgtgtctaaatacattacagttgaaatgactcttaaaaggtggagctgacacaacaccactcagtccacctcattcatttaaccgagatgagacagactgtgttgtggtgagtcaggtggagtaagtaaattggctctactctgtataatcattgctttggcatcagtctctgtattagctgttttgatagttgctattagtaacgaaaccgtagtcagagacataatcgacaaaactatcaacgcttatgctttggatacagaatgttgacaggagaccgtaaacaaggtcttcgaatacgaaacctatgtcttacaactgtcctctggcatttgttccagaagacagtatgaacatgatggaggcagcagcatccgaaccctctcttgatctatgtcatacttgtactacctccgtcgtcgtaggcttgggagagaactagatgataatctgaaactgttggagctgattggccgaggtcgatatggagcagtctattagactttgacaacctcgactaaccggctccagctatacctcgtcaatataaaggctccttggatgagcgtccagttgctgtaaaagtgttttccttatatttccgaggaacctactcgcaggtcaacgacattttcacaaaaggattgcaaaccgtcagaattttatcaacgaaaagaacatttacagagtgcctaacgtttggcagtcttaaaatagttgcttttcttgtaaatgtctcacggattgatggaacatgacaacattgcccgctttatagttggagatgagagagtaactaccttgtactgttgtaacgggcgaaatatcaacctctactctctcacactgcagatggacgcatggaatatttgcttgtgatggagtactatcccagtgacgtctacctgcgtaccttataaacgaacactacctcatgatagggtatggatctttatgcaagtatttaagtctccacacaagtgactgggtaagctacctagaaatacgttcataaattcagaggtgtgttcactgacccattcgtcttgccgtcttgctcattctgttactagaggactggcttatcttcacacagaacggcagaacgagtaagacaatgatctcctgaccgaatagaagtgtgagaattaccacgaggagatcattataaacctgcaatttcccatcgagatttcttaatggtgctcctctagtaatatttggacgttaaagggtagctctaataaacagcagaaatgtcctagtgaaaaatgatggaacctgtgttattagtatttgtcgtctttacaggatcactttttactaccttggacacaataatcagactttggactgtccatgaggctgactggaaatagactggtgcgcccaggctgaaacctgacaggtactccgactgacctttatctgaccacgcgggtccggaggaagataatgcagccataagcgaggttggcactatcagatatatggcctccttctattacgtcggtattcgctccaaccgtgatagtctatatacccaccagaagtgctagaaggagctgtgaacttgagggactgtgaatcagctgtggtcttcacgatcttcctcgacacttgaactccctgacacttagtcgattgaaacaagtagacatgtatgctcttggactaatctattgggagatattaactttgttcatctgtacatacgagaacctgattagataaccctctataatatgagatgtacagacctcttcccaggggaatccgtaccagagtaccagaatactctacatgtctggagaagggtccccttaggcatggtctcatggtcttggcttttcagacagaggttggaaaccatcccacttttgaggatatgcagaccgaaaagtctgtctccaacctttggtagggtgaaaactcctatacgtcgttctcgtgtctagggaaaaacagagacccaagttcccagaagcctggaacaagagcacagatccctttttgtctctgggttcaagggtcttcggaccttagaaaatagcctggcagtgaggtcactcaaggagacaatcgaagactgtttcttttatcggaccgtcactccagtgagttcctctgttagcttctgacaagggaccaggatgcagaggctcggcttactgcacagtgtgctgaggaaaggccctggtcctacgtctccgagccgaatgacgtgtcacacgactcctttccatggctgaacttatgatgatttgggaaagaaacaaatctgtgagcccaactaccgacttgaatactactaaaccctttctttgtttagacactcgggttgagtcaatccaatgtctactgctatgcagaatgaacgcaacctgtcacatatcagttaggttacagatgacgatacgtcttacttgcgttggacagtgtatataggcgtgtgccaaaaattggtccttatccagattattcttcctcctcatatccgcacacggtttttaaccaggaataggtctaataagaaggaggagttacattgaagactctatccatcatactgacagcatcgtgaagaatatttcatgtaacttctgagataggtagtatgactgtcgtagcacttcttataaagctctgagcattctatgtccagcacacctttgactataggggaaaaaaaccgagactcgtaagatacaggtcgtgtggaaactgatatcccctttttttgggaaattcaattaactatgaacgacagcaagcacaagctcgaatccccagcctttaagttaattgatacttgctgtcgttcgtgttcgagcttaggggtcgcctgaaacaagtgtcaccagcctctccaccaacacaacaaccacaaacacggactttgttcacagtggtcggagaggtggttgtgttgttggtgtttgtgcacaggactcacgccaagtactggcatgactactatatctgagatgccatgtgtcctgagtgcggttcatgaccgtactgatgatatagactctacggtaacccagatgaaacaaatctgcataccacaaatgttgcacagtcaattgggtgggtctactttgtttagacgtatggtgtttacaacgtgtcagttaacccccaacccctgtctgcttacagctgacagaagaagacttggaaaccaacaaggttggggacagacgaatgtcgactgtcttcttctgaacctttggttgttgctagacccaaaagaagttgataagaacctcaaggaaagctctgatgagacgatctgggttttcttcaactattcttggagttcctttcgagactactctatctcatggagcactctcttaaacagttcagtggcccagacccactgagctagagtacctcgtgagagaatttgtcaagtcaccgggtctgggtgactcgagtactagttctagcttgctttacccactcataaaacttgcagtagaagctcatgatcaagatcgaacgaaatgggtgagtattttgaacgtcatcttcgaactggacagcaggacttcacacagactgcaaatggccaagcatgtttgattgacctgtcgtcctgaagtgtgtctgacgtttaccggttcgtacaaactttcctgatgttctgcctactcagatctatcctctccccaagcagcagaacaaggactacaagacggatgagtctagataggagaggggttcgtcgtcttgcttcccaagagacctactagtttgcctttgaacaccaaaaattcaacaaagaagggttctctggatgatcaaacggaaacttgtggtttttaagttgtttagagccccggctaaaatttggcagcaagcacaaatcaaacttgaaacaagtctcggggccgattttaaaccgtcgttcgtgtttagtttgaactttgttctcgaaactggagttgccaagatgaatacaatcaatgcagcagaacctcatagctttgacctcaacggttctacttatgttagttacgtcgtcttggagtagtggtgacagtcaccatgaatggtgtggcaggtagaaaccacagtgttaacaccactgtcagtggtacttaccacaccgtccatctttggtgtcacaattctcccatgctgccacaacccaatatgccaataggacagtactatctggccgagggtacgacggtgttgggttatacggttatcctgtcatgatagaccggaaacaaccaacatagtgacacatagggcccaagaaatgttgcagaatcagtttgttggttgtatcactgtgtatcccgggttctttacaacgtcttagtctttattggtgaggacacccggctgaatattaattccagtcctgatgagcaaaataaccactcctgtgggccgacttataattaaggtcaggactactcgttgagcctttactgagacgagagcaacaagctggccatgatgaaggtgttcactcggaaatgactctgctctcgttgttcgaccggtactacttccacaagtggatcgtcttgtggacaggagggaacggccactagaaggtggccgaactacctagcagaacacctgtcctcccttgccggtgatcttccaccggcttgaaattccaataacaacaacagcaatccatgttcagaacaagatgttcttgcttaaggttattgttgttgtcgttaggtacaagtcttgttctacaagaacgacagggtgttccaagcacagcagcagatcctgggccatcaaagcccagaatgtcccacaaggttcgtgtcgtcgtctaggacccggtagtttcgggtcttgagcacagaggcctaattctctggatctttcagccacaaatgtcctggatctcgtgtctccggattaagagacctagaaagtcggtgtttacaggacctaggcagcagtatacagataggtgagtcaacacaagatggcaaatcaggatcccgtcgtcatatgtctatccactcagttgtgttctaccgtttagtcctagaggtgaaaagatcaagaaacgtgtgaaaactccctattctcttaagcggttccacttttctagttctttgcacacttttgagggataagagaattcgccaggcgcccctccacctgggtcatctccactgaatcgctggactgtgaagtcccgcggggaggtggacccagtagaggtgacttagcgacctgacacttcagaacaataatggcagtaacagggcagttcattccaaatccagcactgctgtttgttattaccgtcattgtcccgtcaagtaaggtttaggtcgtgacgacattaccttgcagaaggaggcactgctacaaccatggtgtctaaagatatagaatggaacgtcttcctccgtgacgatgttggtaccacagatttctatatcgaatgaactgtctgtgacttacttgacagacact

SEQ ID NO: 2 - protein, 1080 aa, Human
MTSSLQRPWRVPWLPWTTILLVSTAAASQNQERLCCAFKDPYQQDLGIGESRISHENGTILCSKGSTCYYGLWEKSKGDINLVKQGCCWSHIGDPQECHYEECVVTTTPPSIQNGTYRFCCCCSTDLCNVNFTENFPPPPDTTPLSPPHSFNRDETIIIALASVSVLAVLIVAALCFGYRMLTGDRKQGLHHSMNMMEAAASEPSLDLDNLKLLELIGRGRYGAVVYKGSLDERPVAVKVFSFFANRQNFINEKNIYRVPLMEHDNIARFIVGDERVVTADGRMEYLLVMEYYPNNGSLCKYLSLHTSDWVSSCRLAHSVTRGLAYLHTTELPRGDHYKPAISHRDLLNSRNVLVKNDGTCVISDFGLSMRLTGNRLVRPGGEEDNAAISEVGTIRYMAAPEVLEGAVNLRDCESALKQVDMYALGLIYWEIFFMRCTDLFPGESVPEYQMMAFQTEVGNHPTFEDMQVLVSREKQRPKFPEAWKKENSLAVRSLKETIEDCWWDQDAEARLTAQCAEERMAELMMIWERNKSVSPTTVNPMSTAMQNERNLSHNNRRVPKIGPYPDYSSSSYIEDSIHHTDSIVKNISSEHSMSSTPLTIGEKNRRNSINYERQQAQARIPSPETSVTSLSTNTTTTNTTTGLTPSTGMTTISEMPYYPDETNLHTTNVAQSIGPTPVCLQLTEEDLETNKKLDPKEVDKNLKESSDENNLMEHSLKQFSGPDPLSSTSSSLLYPLIKLAVEAATGQQQDFTQTANGQACLIIPDVLPTQIYPLPKQQNLPKRPTSLPLNTKNSTKKEPRLKFGSKHKSNLKQVVETGVAKMNTINAAEPHVVTVTMNGVAGRNHSVNNSHAATTQYANRTVLSGQQTTNIVTHRAQEMLQNQFIGEDTRLNINSSPDEHHEPLLRREQQAGHDEGVLLDRLVDRRERPLEGGRTNSNNNNSNPCSEQDVLAAQGVPSTAADPGPSKPRRRAQRPNSLDLSATNVLDGSSIQIGESTQDGKSGSSGEKIKKRVKTPYSLKRWWRPSTWVISTESLDCEVNNNGSNRAVHSKSSTAVVTLAEGGTATTMVSKDIGGMNCL

SEQ ID NO: 3 - DNA, 39 bp, Human
gctggtgagtagctccggctttcctttattttagcttcg

SEQ ID NO: 4 - DNA, 39 bp, Human
caaggcaagtgatactttccatattgatttataggatat

SEQ ID NO: 5 - DNA, 39 bp, Human
ctcagtaagtaaagtaacctttgttttcttttaggtcca

SEQ ID NO: 6 - DNA, 39 bp, Human
acaggtaaaaattaccattttcctgttcttataggagac

SEQ ID NO: 7 - DNA, 42 bp, Human
ttggaggtaagtttgccgttattaaaacacttgcagctgatt

SEQ ID NO: 8 - DNA, 42 bp, Human
cccaatgtaagttcttcatagttttcctctatatagggatct

SEQ ID NO: 9 - DNA, 39 bp, Human
ggaggtaagatagtcaataaaattatccaaacagatcat

SEQ ID NO: 10 - DNA, 42 bp, Human
agcgaggtgagtgtatacaaaactctaatttatcaggttggc

SEQ ID NO: 11 - DNA, 39 bp, Human
ccaggtaaaaactactgtctctacaaatccacaggggaa

SEQ ID NO: 12 - DNA, 42 bp, Human
agcctggtaagaaaaaactaatactttgtcttacaggcagtg

SEQ ID NO: 13 - DNA, 39 bp, Human
gaacggtaagaccctaagggctttctttctttaagcaac

SEQ ID NO: 14 - DNA, 39 bp, Human
cagagtaagtggagggatccacttttattttcagtaggt

SEQ ID NO: 15 - protein, 29 aa, Human
LKETIEDCWDQDAEAWLTAQCAEERMAEL

SEQ ID NO: 16 - protein, 29 aa, Homo sapiens
LKETIEDCWDQDAEARLTAQCAEERMAEL

SEQ ID NO: 17 - protein, 29 aa, Mus musculus
LKETIEDCWDQDAEARLTAQCAEERMAEL

SEQ ID NO: 18 - protein, 29 aa, Xenopus laevis
LKETIDDCWDQDAEARLTAQCAEERMAEL

SEQ ID NO: 19 - protein, 29 aa, Gallus gallus
LKETIEDCWDQDAEARLTAQCAEERMAEL

SEQ ID NO: 20 - protein, 29 aa, Home sapiens
VCETLTECWDHDPEARLTAQCVAERFSEL

SEQ ID NO: 21 - protein, 29 aa, Mus musculus
VCETLTECWDHDPEARLTAQCVAERFSEL

SEQ ID NO: 22 - protein, 29 aa, Rattus Norvegicus
VCETLTECWDHDPEARLTAQCVAERFSEL

SEQ ID NO: 23 - protein, 29 aa, Homo sapiens
LCVTIEDCWDHDAEARLSAGCVEERVSLI

SEQ ID NO: 24 - protein, 29 aa, sheep
LCETIEECWDHDAEARLSAGCVGERITQM

SEQ ID NO: 25 - protein, 29 aa, Gallus gallus
LCETIEECWDHDAEARLSAGCVEERIIQM

SEQ ID NO: 26 - protein, 29 aa, Homo sapiens
LRELLEDCWDADPEARLTAECVQQRLAAL

SEQ ID NO: 27 - protein, 29 aa, Rattus Norvegicus
LRELLEDCWDADPEARLTAECVQQRLAAL

SEQ ID NO: 28 - protein, 29 aa, C.elegans
LKKVTEEMWDPEACARITAGCAFARVWNH

SEQ ID NO: 29 - protein, 29 aa, Xenopus laevis
LCVTIEECWDHDAEARLSAGCVEERISQI

SEQ ID NO: 30 - protein, 28 aa, Human
LKETIEDCWDDAEARLTAQCVEERMAEL

(19) United States
(12) Patent Application Publication
(10) Pub. No.: US 2006/0121497 A1
(43) Pub. Date: Jun. 8, 2006

(54) BMPR2 mutations in pulmonary arterial hypertension related to congenital heart disease

(76) Inventors: Jane Morse, Bronx, NY (US); James Knowles, Norwalk, CT (US); Robyn Barst, Scarsdale, NY (US)

Correspondence Address: COOPER & DUNHAM, LLP, 1185 AVENUE OF THE AMERICAS, NEW YORK, NY, 10036, US

(21) Appl. No.: 11/216,721

(22) Filed: Aug. 30, 2005

Related U.S. Application Data
(60) Provisional application No. 60/605,901, filed on Aug. 30, 2004.

(51) Int. Cl. C12Q 1/68

(52) U.S. Cl. 435/6

(57) ABSTRACT

This invention provides a method of detecting whether a subject is predisposed to, or afflicted with, pulmonary arterial hypertension (PAH) which comprises (A) obtaining a suitable sample comprising a nucleic acid encoding bone morphogenetic protein receptor II from the subject; and (B) detecting in the nucleic acid encoding bone morphogenetic protein receptor II whether a mutation is present which is not present in a nucleic acid encoding wildtype bone morphogenetic protein receptor-II. This invention also provides a method of detecting whether a subject is predisposed to, or afflicted with, pulmonary arterial hypertension (PAH) which comprises (A) obtaining a suitable sample comprising bone morphogenetic protein receptor II from the subject; and (B) detecting in the bone morphogenetic protein receptor II whether a mutation is present which is not present in wildtype bone morphogenetic protein receptor-II.

This application claims benefit of U.S. Provisional No. 60/605,901, filed Aug. 30, 2004, the contents of which are hereby incorporated by reference into this application.

The invention disclosed herein was made with U.S. Government support under Grant No. HL60056 from the National Institute of Health, Department of Health and Human Services. Accordingly, the United States Government has certain rights in this invention.

Throughout this application, certain publications are referenced. Full citations for these publications may be found immediately preceding the claims. The disclosures of these publications are hereby incorporated by reference into this application in order to more fully describe the state of the art to which this invention relates.

### BACKGROUND OF THE INVENTION

Pulmonary arterial hypertension (PAH) consists of a group of vascular abnormalities with elevated pulmonary arterial pressure and pulmonary vascular resistance. The clinical spectrum includes familial and sporadic idiopathic PAH (IPAH), previously referred to as primary pulmonary hypertension, as well as PAH related to congenital heart disease (CHD), portal hypertension, connective tissue diseases, HIV-infection, and appetite suppressant exposure. Germline mutations of bone morphogenetic protein receptor 2 (BMPR2), a member of the TGF-B superfamily, have been found in familial and sporadic forms of IPAH, and in appetite-suppressant PAH but not in PAH with HIV-infection or PAH with connective tissue diseases.

BMPR2 mutations have not been previously reported in PAH patients with CHD (PAH/CHD) in whom the PAH is due to pulmonary vascular obstructive disease. The natural history of CHD associated with large systemic to pulmonary shunts (e.g. atrial and ventricular septal defects, patent ductus arteriosus) results in pulmonary vascular obstructive disease, i.e. the Eisenmenger syndrome (ES). Approximately one third of all patients with CHD who do not undergo early “corrective” surgery, or who die from other causes, will die from pulmonary vascular disease. Although the pathophysiologic mechanisms, which lead to the histopathologic changes seen in ES, are not completely understood, CHD repaired within the first two years of life is unlikely to lead to pulmonary vascular disease. It is unclear in certain patients with CHD whether the PAH results from increased flow, a “primary” pulmonary vascular abnormality, or both.

Members of the TGF-B/BMP signaling pathway are particularly important in vasculogenesis and embryonic heart development. Heterodimers of BMPR2 form a heterotetramer with type 1 receptors, BMPR1a (ALK3) and BMPR1b (ALK6), in the presence of a BMP ligand such as BMP2 or BMP4. Mice with tissue specific inactivation of ALK3 (BMPR1a) have abnormal endocardial cushion morphogenesis. BMPR2 has been implicated in abnormal septation in the mouse resulting in a conotruncal abnormality, i.e. truncus arteriosus. Jiao and coworkers reported that cardiac muscle conditional knock-out of BMP4 resulted in reduced atrioventricular septation.

### SUMMARY OF THE INVENTION

This invention provides a method of detecting whether a subject is predisposed to, or afflicted with, pulmonary arterial hypertension (PAH) which comprises (A) obtaining a suitable sample comprising a nucleic acid encoding bone morphogenetic protein receptor II from the subject; and (B) detecting in the nucleic acid encoding bone morphogenetic protein receptor II whether a mutation is present which is not present in a nucleic acid encoding wildtype bone morphogenetic protein receptor-II, wherein the mutation described relative to a difference from the sequence encoding wildtype bone morphogenetic protein receptor II set forth in SEQ ID NO:1 is selected from the group consisting of: (1) a substitution of an adenosine nucleotide located at position 125 with a guanosine nucleotide; (2) a substitution of a guanosine nucleotide located at position 140 with an adenosine nucleotide; (3) a substitution of an adenosine nucleotide located at position 304 with a guanosine nucleotide; (4) a substitution of a thymidine nucleotide located at position 319 with a cytosine nucleotide; (5) a substitution of an adenosine nucleotide located at position 556 with a guanosine nucleotide; (6) a substitution of an adenosine nucleotide located at position 1509 with a cytosine nucleotide; wherein the presence of such a mutation indicates that the subject is predisposed, to or afflicted with, pulmonary arterial hypertension (PAH).

This invention also provides a method of detecting whether a subject is predisposed to, or afflicted with, pulmonary arterial hypertension (PAH) which comprises (A) obtaining a suitable sample comprising bone morphogenetic protein receptor II from the subject; and (B) detecting in the bone morphogenetic protein receptor II whether a mutation is present which is not present in wildtype bone morphogenetic protein receptor-II, wherein the mutation described relative to a difference from the wildtype bone morphogenetic protein receptor II sequence set forth in SEQ ID NO:2 is selected from the group consisting of: (1) a substitution of a glutamine residue located at position 42 with an arginine residue; (2) a substitution of a glycine residue located at position 47 with an asparagines residue; (3) a substitution of a threonine residue located at position 102 with an alanine residue; (4) a substitution of a serine residue located at position 107 with a proline residue; (5) a substitution of a methionine residue located at position 186 with a valine residue; (6) a substitution of a glutamic acid residue located at position 503 with an aspartic acid residue; wherein the presence of such a mutation indicates that the subject is predisposed, to or afflicted with, pulmonary arterial hypertension (PAH).

### BRIEF DESCRIPTION OF THE FIGURES

FIG. 1

Intron/Exon boundaries of the human BMPR2 gene. The nucleotide sequence of all intron/exon boundaries and the known size of each exon and approximate size of each intron are shown.

FIG. 2

Intron/Exon structure of the human BMPR2 gene. Intron and exon sizes are as indicated. Mutations that cause premature termination of BMPR2 are shown as closed arrows. Open arrows indicate mutations in Arg491. The transmembrane and kinase domains are encoded by the indicated exons.

FIG. 3

Nucleic acid and amino acid sequences for wildtype BMPR2. The nucleic acid sequence is also set forth in SEQ ID NO:1. The amino acid sequence is also set forth in SEQ ID NO:2.

FIG. 4

BMPR2 mutations observed in PPH. DNA sequences are referenced to GENEBANK BMPR2 cDNA sequence number NM_{—}001204. These sequences are also set forth in FIG. 5. #A/#C/#U is the number of affected known carrier or unaffected individuals in each family or set of families. Nomenclature: fs denotes a frameshift mutation; X1 denotes a one amino acid tail after the frameshift.

FIG. 5

Nucleic acid and amino acid sequences for wildtype BMPR2. The nucleic acid sequence is also set forth in SEQ ID NO:1. The amino acid sequence is also set forth in SEQ ID NO:2.

### DETAILED DESCRIPTION OF THE INVENTION

This invention provides a method of detecting whether a subject is predisposed to, or afflicted with, pulmonary arterial hypertension (PAH) which comprises (A) obtaining a suitable sample comprising a nucleic acid encoding bone morphogenetic protein receptor II from the subject; and (B) detecting in the nucleic acid encoding bone morphogenetic protein receptor II whether a mutation is present which is not present in a nucleic acid encoding wildtype bone morphogenetic protein receptor-II, wherein the mutation described relative to a difference from the sequence encoding wildtype bone morphogenetic protein receptor II set forth in SEQ ID NO:1 is selected from the group consisting of: (1) a substitution of an adenosine nucleotide located at position 125 with a guanosine nucleotide; (2) a substitution of a guanosine nucleotide located at position 140 with an adenosine nucleotide; (3) a substitution of an adenosine nucleotide located at position 304 with a guanosine nucleotide; (4) a substitution of a thymidine nucleotide located at position 319 with a cytosine nucleotide; (5) a substitution of an adenosine nucleotide located at position 556 with a guanosine nucleotide; (6) a substitution of an adenosine nucleotide located at position 1509 with a cytosine nucleotide; wherein the presence of such a mutation indicates that the subject is predisposed, to or afflicted with, pulmonary arterial hypertension (PAH).

In one embodiment, the subject is human. In another embodiment, the subject has a congenital heart defect.

One skilled in the art would know various methods for detecting a mutation on the nucleic acid level. For example, one may use a nucleic acid probe which binds to a target nucleic acid. Such nucleic acid probe may be one which is detectable. For example, the detectable nucleic acid may be labeled with a detectable marker. Such markers include but are not limited to a radioactive, a calorimetric, a luminescent, and a fluorescent label. For example, the probe may be specific for a sequence having a particular mutation (such as one of the mutations described herein), such that it is capable of detecting a mutant nucleic acid. Alternatively, the probe may specific for a corresponding wildtype sequence for a particular mutation, such that it is capable of detecting a wildtype nucleic acid (i.e. one which is wildtype with respect to the mutation).

In various embodiments, the nucleic acid probe included but is not limited to nucleic acids which are at least 5, nucleotides in length, at least 10, nucleotides in length, at least 15, nucleotides in length, at least 20 nucleotides in length, at least 25 nucleotides in length, an at least 30 nucleotides in length. The subject invention also encompasses other lengths of nucleic acid probes. In one embodiment the nucleic acid and/or nucleic acid probe is DNA. In another embodiment the nucleic acid and/or nucleic acid probe is RNA.

One skilled in the art would know various conditions under which the hybridization may take place. For example, high stringency hybridization conditions may be selected at about 5° C. lower than the thermal melting point (Tₘ) for the specific sequence at a defined ionic strength and pH. The Tₘ is the temperature (under defined ionic strength and pH) at which 50% of the salt concentration is at least about 0.02 molar at pH 7 and the temperature is at least about 60° C. As other factors may significantly affect the stringency of hybridization, including, among others, base composition and size of the complementary strands, the presence of organic solvents, i.e. salt or formamide concentration, and the extent of base mismatching, the combination of parameters is more important than the absolute measure of any one. For example, high stringency may be attained by overnight hybridization at about 68° C. in a 6×SSC solution, washing at room temperature with 6×SSC solution, followed by washing at about 68° C. in a 0.6×SSC solution.

Hybridization with moderate stringency may be attained for example by: 1) filter pre-hybridizing and hybridizing with a solution of 3×SSC, 50% formamide, 0.1M Tris buffer at pH 7.5, 5× Denhardt's solution; 2.) pre-hybridization at 37° C. for 4 hours; 3) hybridization at 37° C. with amount of labeled probe equal to 3,000,000 cpm total for 16 hours; 4) wash in 4×SSC and 0.1% SDS solution; 5) wash 4× for 1 minute each at room temperature in 4×SSC at 60° C. for 30 minutes each; and 6) dry and expose to film.

Nucleic acid probe technology is well known to those skilled in the art who readily appreciate that such probes may vary greatly in length and may be labeled with a detectable label, such as a radioisotope or fluorescent dye, to facilitate detection of the probe. DNA probe molecules may be produced by insertion of a DNA molecule having the full-length or a fragment of the isolated nucleic acid molecule of the DNA virus into suitable vectors, such as plasmids or bacteriophages, followed by transforming into suitable bacterial host cells, replication in the transformed bacterial host cells and harvesting of the DNA probes, using methods well known in the art. Alternatively, probes may be generated chemically from DNA synthesizers.

RNA probes may be generated by inserting the full length or a fragment of the isolated nucleic acid molecule of the DNA virus downstream of a bacteriophage promoter such as T3, T7 or SP6. Large amounts of RNA probe may be produced by incubating the labeled nucleotides with a linearized isolated nucleic acid molecule of the DNA virus or its fragment where it contains an upstream promoter in the presence of the appropriate RNA polymerase.

As defined herein nucleic acid probes may be DNA or RNA fragments. DNA fragments can be prepared, for example, by digesting plasmid DNA, or by use of PCR, or synthesized by either the phosphoramidite method described by Beaucage and Carruthers, 1981, Tetrahedron Lett. 22, 1859-1862 or by the triester method according to Matteucci et al., 1981, Am. Chem. Soc. 103:3185. A double stranded fragment may then be obtained, if desired, by annealing the chemically synthesized single strands together under appropriate conditions or by synthesizing the complementary strand using DNA polymerase with an appropriate primer sequence. Where a specific sequence for a nucleic acid probe is given, it is understood that the complementary strand is also identified and included. The complementary strand will work equally well in situations where the target is a double-stranded nucleic acid. It is also understood that when a specific sequence is identified for use a nucleic probe, a subsequence of the listed sequence which is 25 base pairs (bp) or more in length is also encompassed for use as a probe.

Another way to detect a mutation on the nucleic acid level is to perform nucleic acid sequencing on the nucleic acid sample obtained from the subject. For example, one may perform DNA sequencing to detect the presence of the mutation. One skilled in the art knows how to sequence a particular nucleic acid. Examples of such sequencing methods include The Maxam and Gilbert method and the Sanger method, both of which are described in Recombinant DNA, Second Edition by James Watson et al (1992), Scientific American Books the contents of which are hereby incorporated by reference.

If a particular mutation results in the gain and/or loss of particular restriction cleavage sites, one may also perform a restriction digest on the nucleic acid to determine if a particular mutation is present.

This invention also provides a method of detecting whether a subject is predisposed to, or afflicted with, pulmonary arterial hypertension (PAH) which comprises (A) obtaining a suitable sample comprising bone morphogenetic protein receptor II from the subject; and (B) detecting in the bone morphogenetic protein receptor II whether a mutation is present which is not present in wildtype bone morphogenetic protein receptor-II, wherein the mutation described relative to a difference from the wildtype bone morphogenetic protein receptor II sequence set forth in SEQ ID NO:2 is selected from the group consisting of: (1) a substitution of a glutamine residue located at position 42 with an arginine residue; (2) a substitution of a glycine residue located at position 47 with an asparagines residue; (3) a substitution of a threonine residue located at position 102 with an alanine residue; (4) a substitution of a serine residue located at position 107 with a proline residue; (5) a substitution of a methionine residue located at position 186 with a valine residue; (6) a substitution of a glutamic acid residue located at position 503 with an aspartic acid residue; wherein the presence of such a mutation indicates that the subject is predisposed, to or afflicted with, pulmonary arterial hypertension (PAH).

In one embodiment, the subject is human. In another embodiment, the subject has a congenital heart defect.

For example, to detect the presence of a mutation, one may use a detectable antibody capable of binding to an epitope which is present in the mutant protein but not present in the wildtype protein. The binding of the antibody to the protein indicates a mutant protein and therefore, that the subject is predisposed to or afflicted with the pulmonary disease. One may use a detectable antibody capable of binding to an epitope which is present in the wildtype protein but not present in the mutant protein. Binding of the antibody to the protein indicates a wildtype protein.

If a mutation results in a nonconservative mutation, such as a positively charged amino acid for a negatively charged amino acid, such mutation may be identified by running the protein in a gel to determine a difference in charge. One skilled in the art would know how to identify a nonconservative mutation.

For mutations which result in truncated proteins, such as the introduction of a stop codon or a frameshift mutation which results in a truncation, such mutations may be identified by detecting the truncated protein, such as by running the protein on a gel and determining its size based on the distance that it moves within the gel.

As used herein, “subject” means any animal or artificially modified animal. The subjects include but are not limited to a human being, a primate, an equine, an opine, an avian, a bovine, a porcine, a canine, a feline or a mouse. Artificially modified animals include, but are not limited to, SCID mice with human immune systems. The animals include but are not limited to mice, rats, dogs, guinea pigs, ferrets, rabbits, and primates. In the preferred embodiment, the subject is a human being.

As used herein, the following standard abbreviations are used throughout the specification to indicate specific amino acids: A=ala=alanine; R=arg=arginine; N=asn=asparagine D=asp=aspartiic acid; C=cys=cysteine; Q=gln=glutamine; E=glu=glutamic acid; G=gly=glycine; H-his=histidine; I=ile=isoleucine;

L=leu=leucine; K=lys=lysine; M=met=methionine; F=phe=phenylalanine; P=pro=proline; S=ser=serine; T=thr=threonine; W=trp=tryptophan; Y=tyr=tyrosine; and V=val=valine.

As used herein, the following standard abbreviations are used throughout the specification to indicate specific nucleotides: C=cytosine; A=adenosine; T=thymidine; and G=guanosine.

This invention is illustrated in the Experimental Details section which follows. This section is set forth to aid in an understanding of the invention but is not intended to, and should not be construed to limit in any way the invention as set forth in the claims which follow thereafter.

### EXPERIMENTAL DETAILS

First Series of Experiments

Familial Primary Pulmonary Hypertension (Familial PPH) is a rare autosomal dominant disorder with reduced penetrance that has been mapped to a 3-centimorgan region on chromosome 2q34 (PPH1 locus). The phenotype is characterized by monoclonal plexiform lesions of proliferating endothelial cells in pulmonary arterioles that lead to elevated pulmonary artery pressures, right ventricular failure, and death. Although PPH is rare, cases secondary to known etiologies are more common and include those associated with the appetite suppressant drugs, including phentermine-fenfluramine. Thirty five multiplex families with the disorder were genotyped using 27 microsatellite markers, disease haplotypes were constructed, and evidence of haplotype sharing across families using the program TRANSMIT was observed. Suggestive evidence of sharing was observed using markers GGAA19e07 and D2S307, and three nearby candidate genes were examined by dHPLC using individuals from 19 families. One of these genes (BMPR2), which encodes the bone morphogenetic protein receptor-II (BMPR-II), was found to contain 5 mutations that predict premature termination of the protein product and 2 missense mutations. These mutations were not observed in 196 control chromosomes. These findings indicate that the BMP signaling pathway is defective in patients with PPH and may implicate the pathway in the non-familial forms of the disease.

A number of multiplex families with PPH were collected using experimental protocols approved by the Institutional Review Board of Columbia University College of Physicians and Surgeons. Methods used for clinical examination, as well as the diagnostic criteria, have been described elsewhere (Morse et al. 1997). Using DNA that was extracted from whole-blood samples or formalin-fixed, paraffin-embedded tissue, 35 of these families (72 affected and 319 normals and carriers) were genotyped using 27 microsatellite markers located in the 3-centimorgan minimal genetic region as previously described (Deng et al. 2000). Using the genetic model and marker order determined by radiation hybrid mapping (Deng et al. 2000), a 10-marker multipoint analysis using GENEHUNTER 2.0 (Kruglyak et al. 1996), gave a nearly constant lod score of 10 across the region (data not shown). The maximum lod scores of the 2-point analyses, using MLINK from FASTLINK v4.1p (Cottingham, Jr., Idury, and Schaffer 1993), at a recombination fraction of zero were more variable, ranging from 0.6 to 8.6, with the higher scores clustering towards the telomeric end (data not shown). Given the low prevalence of the disorder and that some of the families were from a common founder, the 27-marker microsatellite disease haplotype from each was reconstructed when possible and visually inspected for shared segments. No obvious shared DNA segments were found, so the haplotype analysis program, TRANSMIT v2.5 1999 (Clayton 1999), was used to look in a more rigorous fashion. Suggestive evidence of sharing (p=0.07) was found with the 345/214 base pair haplotype of markers GGAA19e07 and D2S307. Since these markers were in the telomeric cluster the mutation scan was begun in this region.

The genetic variation in the coding sequence of three nearby candidate genes was investigated by examining in 22 individuals from the 19 FPPH families and 2 normal controls using dHPLC with a WAVE® Nucleic Acid Fragment Analysis System from Transgenomics, Inc. (Omaha, Nebr.), as per the manufacturers directions and as described (Underhill et al. 1997; O'Donovan et al. 1998) These individuals were chosen on the basis of the amount of DNA available. PCR amplification products (max size=602 bp) were run with up to three melting profiles for fragments with multiple melting domains. DNA sequence determination of fragments containing potential variants were performed by cycle sequencing using Big Dye terminators from Applied BioSystems Inc., (Foster City, Calif.) and sequencing products were resolved on Long Ranger gels (Biowhittaker Molecular Applications, Rockland, Me.) and detected with an ABI Model 377 DNA sequencer (ABI, Foster City, Calif.). DNA sequence traces were analyzed using Vector NTI suite 5.5 (Informax Inc., Bethesda, Md.). The first two genes, CD28 and CTLA4, were candidates due to their involvement in immune system regulation (Morse and Barst 1994). No variation in CD28 was observed. In CTLA4, one previously unreported SNP (49A>G) with an allele frequency of 0.50 that causes a non-conservative change in protein structure (A17T) was found. It was homozygous in some of the patients and one of the controls and was ruled out as a potential disease mutation.

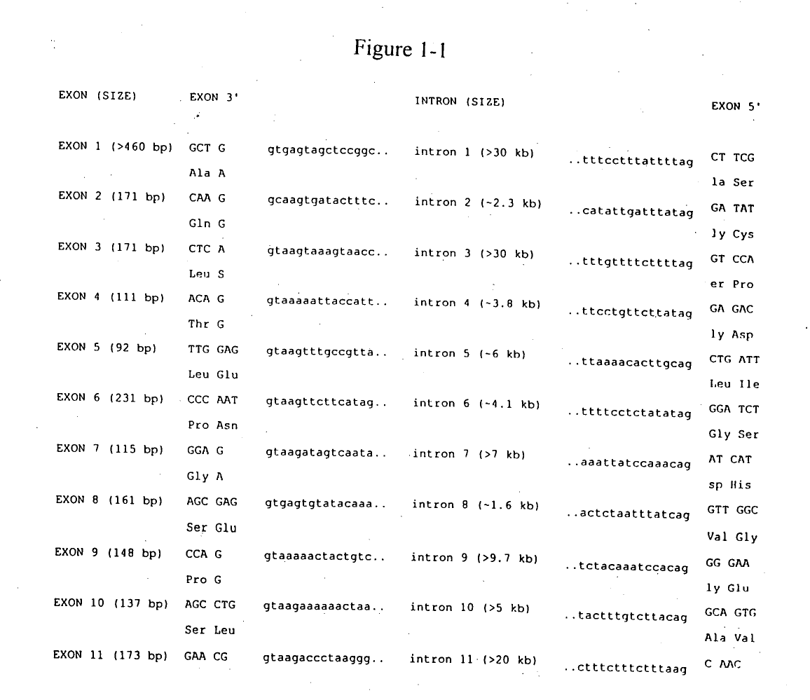

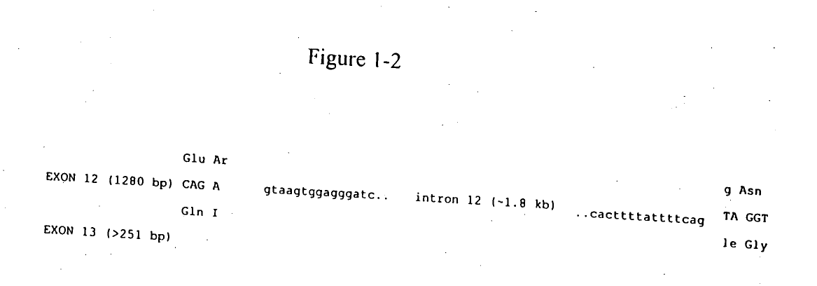

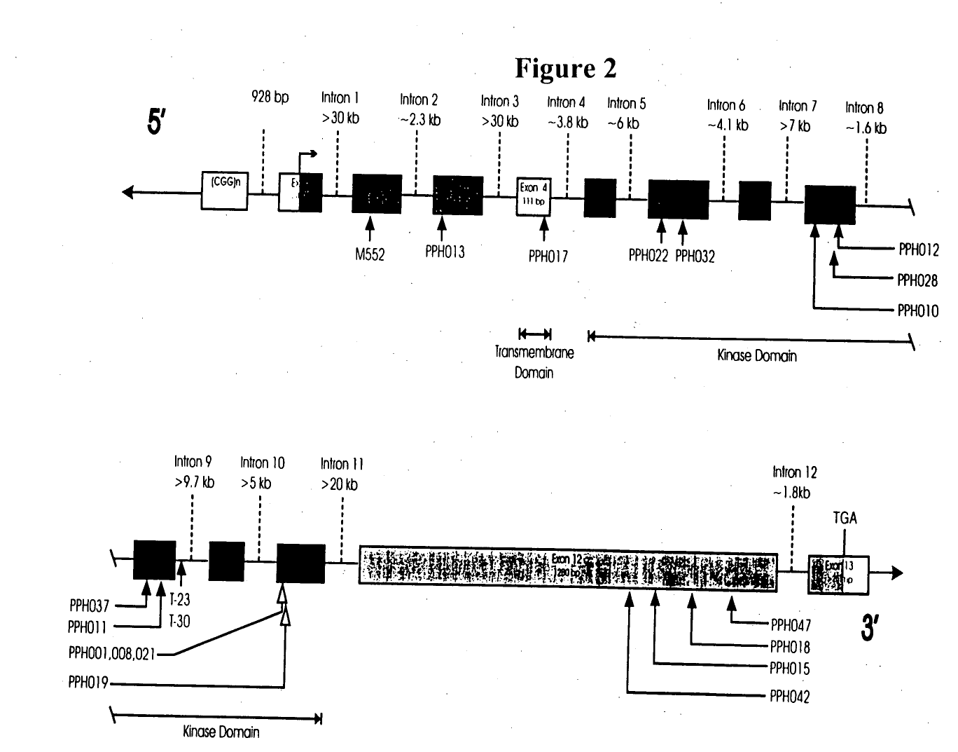

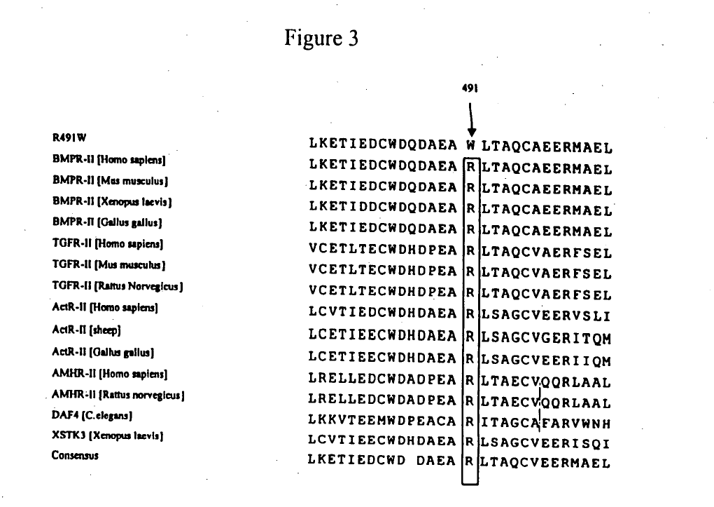

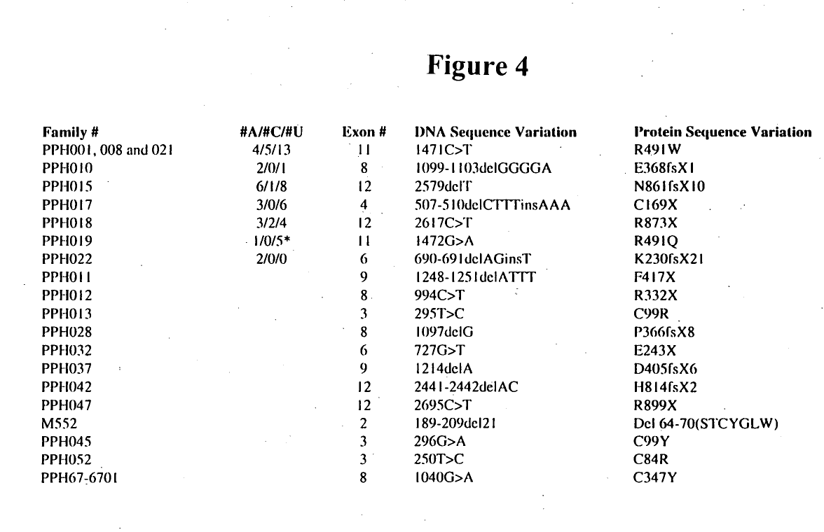

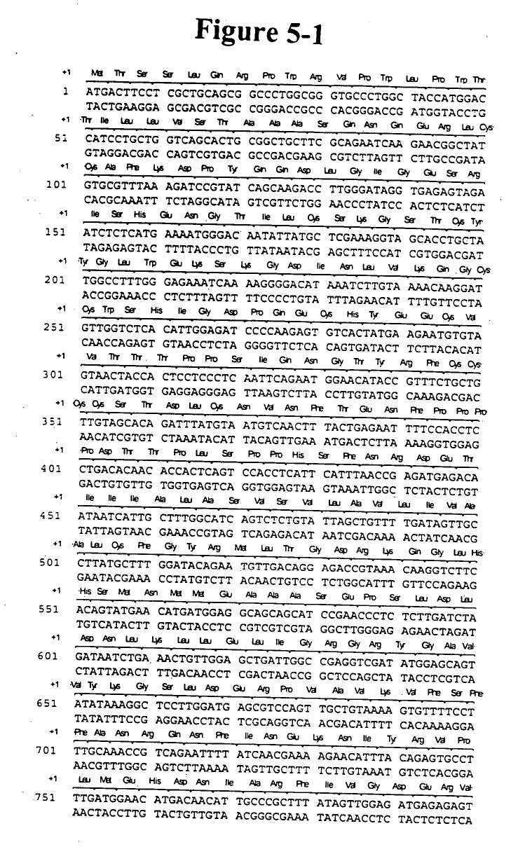

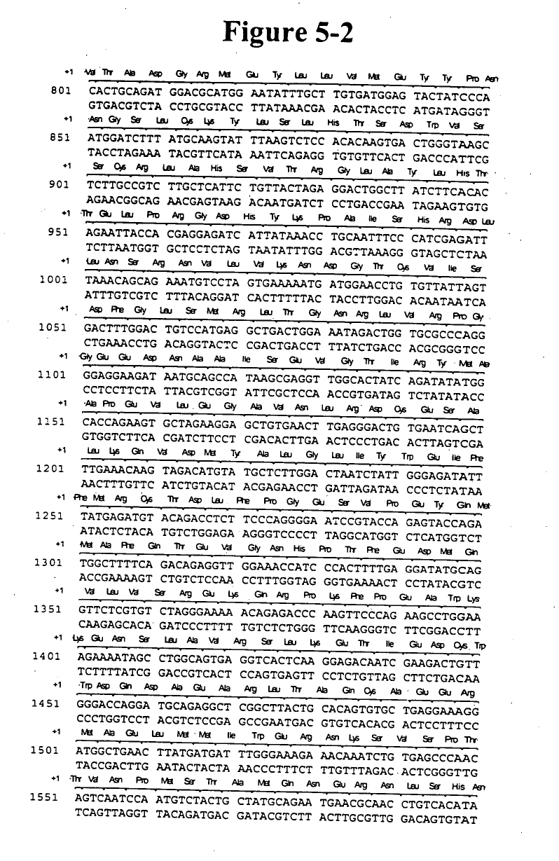

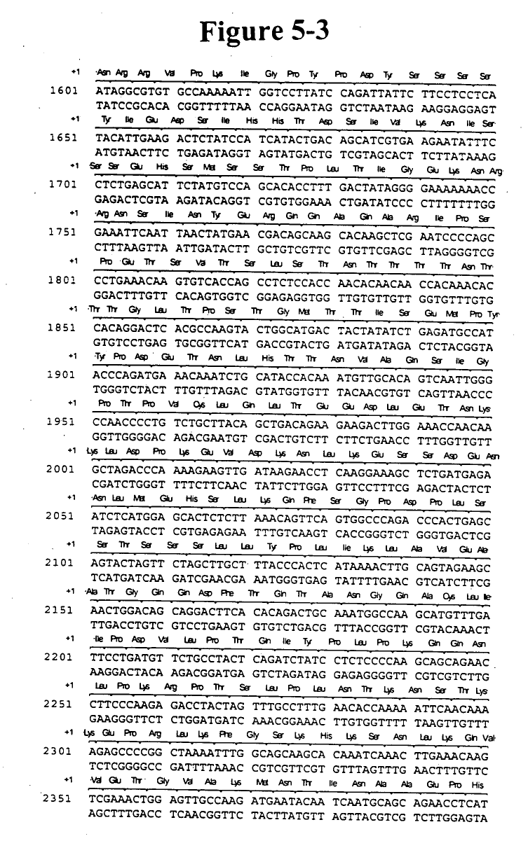

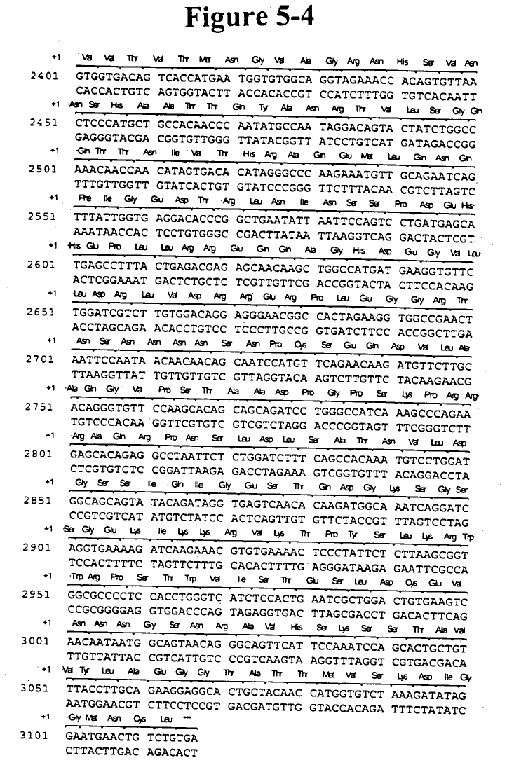

The third positional candidate, the gene encoding the bone morphogenetic protein receptor-II (BMPR2, also known as T-ALK, CL4-1 and BRK-3), a member of the TGF-_receptor superfamily, was suggested by the role of the BMP signaling pathway in lung morphogenesis (Warburton et al. 2000). The cDNA sequence of this approximately 4 kilobase gene encoding a 1038 amino acid protein had been previously described (Kawabata, Chytil, and Moses 1995; Liu et al. 1995; Rosenzweig et al. 1995; Nohno et al. 1995). To deduce the genomic structure of BMPR2 (FIGS. 1 and 2) homologous genomic sequences to exons 1, and 8-13 were found by querying the NCBI high-throughput genome sequence (HTGS) database using BLAST (Altschul et al. 1990). The intron size and DNA sequence of the other intron-exon boundaries were determined by amplifying and sequencing PCR products using oligonucleotide primers designed to amplify across neighboring exons, or out to a nearby Alu repeats, using the structure of mouse BMPR2 (Beppu et al. 1997) as a guide. Oligonucleotide primers were then designed to amplify the exons from genomic DNA of the patients. These PCR fragments were screened by dHPLC and the DNA sequence of those containing apparent variation was determined. Mutations that are likely to disrupt the function of the receptor in 9 of the 19 families screened were observed. Five of these predict premature termination of BMPR-II in exons 4, 6, 8 and 12, and each was only seen in one family (FIG. 2). In addition, a SNP in exon 11 that causes a non-conservative change in amino acid sequence, from an arginine, conserved in all known type II TGF-_superfamily receptors (FIG. 3), to tryptophan was seen in three families (FIG. 2). The same arginine was changed to glutamine in another family (PPH019), but both parents were genotypically normal. The observation of this new mutation suggests that sporadic cases of PPH might also be caused by mutations in BMPR2. Except for this family, the expected pattern of mutations was observed when all additional members of the other 8 families were screened using dHPLC and DNA sequencing. None of the putative mutations were observed in 96 additional samples (196 chromosomes total). Applying Fisher's exact test to the data for all nine mutations, a significant difference (p-value <0.0001) in mutation rate between cases and controls was observed, as well as a synonymous SNP (2811G>A) with a minor allele frequency of 21% in both samples.

The mutation in exon 4 is in the transmembrane domain and those in exons 6, 8 and 11 are in the kinase domain of this serine/threonine kinase receptor (FIG. 2). By analogy to studies of the T_R-II gene product (Wieser et al. 1993), at least three of these mutations (exons 4, 6 and 8) should encode a non-functional receptor that is unable to phosphorylate a type-I receptor and propagate the signal from a BMP ligand. The two mutations in exon 11 change Arg491. Since it is highly conserved and arginine is the most frequently changed amino acid in disease mutations (Human Gene Mutation Database), Arg491 is probably important to the function of BMPR-II. The mutations in exon 12 occur in the intracellular C-terminal domain of unknown function that is unique to BMPR-II.

The entire publicly available coding sequence of BMPR2 was screened, but no causative mutation was found in 10 of the 19 families. The microsatellite data are consistent, but not conclusive, with linkage to PPH1 in all 19, but it is possible that the families with little linkage information could be unlinked to 2q34 and may have mutations in other genes in the BMP signaling pathway. However, several of the linked families are large (individual lod scores >2), suggesting that the entire gene has not been screened. mRNA transcripts of 5, 6.5, 8 and 11.5 kb have been observed on Northern blots, with the longest transcript predominating in lung (Kawabata, Chytil, and Moses 1995; Rosenzweig et al. 1995; Nohno et al. 1995), so some alternatively spliced exons may have been missed in the screen. In addition, there is a (GGC)₁₂ trinucleotide repeat at the 5′ end of the gene, at positions −928 to −963. This repeat is polymorphic in our families.

So how do these mutations cause. PPH? It is unlikely that they act as a dominant-negative by inhibiting apoptotic effect of the TGF pathway because BMPR-II does not associate with type-I receptors of the TGF-_family in transient expression assays using mammalian cells (Liu et al. 1995), even though this occurs in vitro (Kawabata, Chytil, and Moses 1995; Liu et al. 1995; Nohno et al. 1995). As would be predicted from what is known about the role of the BMP signaling pathway in early development, mice homozygous for a mutation in the kinase domain of BMPR2 die at day 9.5, prior to gastrulation (heterozygotes are grossly normal) (Beppu et al. 2000), so this function of the pathway must be functioning in patients with PPH. The BMP pathway induces apoptosis in some cell types (Soda et al. 1998; Kimura et al. 2000), so a partial block of signal transmission by haplo-insufficiency of BMPR-II might have a slow proliferative effect. BMP signaling may occur through both the Smad (Massague 1998) and mitogen-activated protein kinase (MAPK) (Kimura et al. 2000) cascades and both are inhibited by Smad6, which can be induced by vascular shear stress (Topper et al. 1997). The reduced apoptotic signals from the BMP pathway, caused by either mutations in BMPR2, other molecules in the signaling cascades (by analogy to hereditary hemorrhagic telangiectasia (Massague 1998)), or shear stress via Smad6, possibly after an initial nidus of vascular injury, might underlie many forms of PPH, including those associated with HIV or appetite suppressant drugs.

Second Series of Experiments

Material and Methods

Study subjects: All studies and procedures were approved by the Columbia Presbyterian Medical Center Institutional Review Board, Columbia University, New York, N.Y. and comply with the Declaration of Helsinki. The study group consisted of two cohorts, 66 children (ages <18 y) and 40 adults (18 y and older) with PAH/CHD. The diagnosis of each CHD was determined echocardiographically. The diagnosis of PAH due to pulmonary vascular obstructive disease was confirmed by right heart catheterization demonstrating mean pulmonary arterial pressure >25 mm Hg, mean pulmonary capillary wedge pressure <15 mmHg and pulmonary vascular resistance index >3 Wood units·m². All patients underwent acute vasodilator drug testing during the right heart catheterization. Evaluation and work-up excluded other causes of PAH, such as IPAH, PAH with HIV-infection, connective tissue diseases or appetite-suppressant drug exposure. None of the patients reported a family history of PAH.

Mutational analysis of BMPR2: The 13 exons and flanking intron sequences of BMPR2 were mutation screened by denaturing high-pressure liquid chromatography (dHLPC, Transgenomics, Inc.) as previously described. All samples with inconclusive or mutation-suggestive dHPLC results were sequenced bi-directionally using the Big Dye® Terminator v1.1 Cycle Sequencing Kit (Applied Biosystems, Foster City, Calif.), using an ABI 3100. Mutation analysis was performed blind of a patient's diagnosis with the aid of Mutation Surveyor v2.0 (SoftGenetics Inc.).

Results

Table 1 (below) illustrates the category of CHD, the number of defects repaired, and the presence of BMPR2 mutations in the 40 adults and 66 children with PAH/CHD. All patients were non-responders with acute vasodilator testing. The predominant CHD in both cohorts were atrial and ventricular septal defects; with the majority being unrepaired. None of the patients had primum atrial septal defects.

TABLE 1

| | Adults | | | Children | | |
|---|---|---|---|---|---|---|
| | N=40 | | | N=66 | | |
| | Total | | BMPR2 | Total | | BMPR2 |
| | # | Repaired | N=3 | # | Repaired | N=3 |
| Patent ductus | 2 | 0 | 0 | 6 | 3 | 0 |
| arteriosus | | | | | | |
| Atrial septal | 17 | 7 | 0 | 21 | 5 | 1* |
| defect (ASD) | | | | | | |
| ASD/Partial | 3 | 0 | 0 | 0 | 0 | 1* |
| anomalous | | | | | | |
| pulmonary | | | | | | |
| venous return | | | | | | |
| (PAPVR) | | | | | | |
| Ventricular | 8 | 1 | 0 | 15 | 8 | 0 |
| septal defect | | | | | | |
| PAPVR | 1 | 0 | 0 | 3 | 2 | 0 |
| Transposition | 3 | 3 | 0 | 7 | 3 | 0 |
| of the great | | | | | | |
| vessels | | | | | | |
| Atrial | 4 | 1 | 3 | 6 | 3 | 0 |
| ventricular | | | | | | |
| canal | | | | | | |
| Rare | 2 | 0 | 0 | 8 | 5 | 1 |
| Total # | 40 | 12 | 3 | 66 | 29 | 3 |

Category of CHD in 40 Adults and 66 Children with PAH, Number of Repaired CHD, and Presence of BMPR2 Mutations. Both CHDs had patent ductus arteriosus.

Table 2 (below) illustrates the clinical and hemodynamic findings and the type of BMPR2 mutation present in the six mutation-positive patients. Strikingly, most of the BMPR2 mutations observed in the adult cohort occurred in patients with atrioventricular canals (also referred to as endocardial cushion defects) and these mutations were found in 3 of the 4 (75%) of such cases. One of these patients (#1, Table 2) also had Down syndrome. The range of defects was more variable in the children. One had an atrial septal defect and patent ductus arteriosus (who also had a ring 14 chromosome, #4 Table 2), one an atrial septal defect, patent ductus arteriosus and partial anomalous pulmonary venous return and one an aortopulmonary window and a ventricular septal defect. None of the six children with atrioventricular canal type C had BMPR2 mutations. Five of these six children also had Down syndrome (not illustrated).

TABLE 2
Clinical and Hemodynamic Findings and BMPR2 Mutations
in BMPR2-positive Children and Adults with PAH/CHD

| Subject | 1, adult | 2, adult | 3, adult | 4, child | 5, child | 6, child |
|---|---|---|---|---|---|---|
| Age initial | 19 | 57 | 19 | 3 | 13 | 3.5 |
| cath (y) | | | | | | |
| Age at PAH | 1 y | 16 y | 5 y | 3 y | 2 y | 19 m |
| Diagnosis | | | | | | |
| Age at Repair | n/a | n/a | 4 | n/a | n/a | 20 m |
| (y) | | | | | | |
| Outcome/Age | Lost to | Lost to | Tx, 23 | A, 16 | A, 18 | D, 5 |
| (y) | f/u | f/u | | | | |
| Sex (M/F) | F | F | M | F | M | M |
| Ethnicity | White | White | White | White | Asian | White |
| Type of CHD | Atrial | Atrial | Atrial | Atrial septal | Atrial | Aorto- |
| | ventricular | ventricular | ventricular | defect/Patent | septal | pulmonary |
| | canal, C | canal, C | canal, C | ductus | defect/ | Window and |
| | | | | arteriosus | Patent | Ventricular |
| | | | | | ductus | septal |
| | | | | | arteriosus/ | defect |
| | | | | | Partial | |
| | | | | | anomalous | |
| | | | | | pulmonary | |
| | | | | | venous | |
| | | | | | return | |
| PAPm (mmHg) | 70 | n/d | 75 | 61 | 69 | 75 |
| RAPm (mmHg) | 7 | n/d | 8 | 4 | 0 | 4 |
| CI (L/min/m²) | 8.3 | n/d | 4.0 | 2.3 | 3.2 | 4.0 |
| PVR (Wood | 18 | n/d | 23 | 19 | 10 | 22 |
| units• m²) | | | | | | |
| SVR (Wood | 8 | n/d | 13 | 25 | 21 | 13 |
| units• m2) | | | | | | |
| MVSaO₂ (%) | 50 | n/d | 52 | 65 | 60 | 68 |
| SaO₂ (%) | 64 | n/d | 84 | 91 | 86 | 90 |
| Genetic | Down | N | N | Ring 14 | N | N |
| Syndrome | | | | | | |
| BMPR2 Mutation | | | | | | |
| Exon | 2 | 3 | 3 | 5 | 11 | 2 |
| Nucleic Acid | 125A>G | 304A>G | 319T>C | 556A>G | 1509A>C | 140G>A |
| Change | | | | | | |
| Amino Acid | Q42R | T102A | S107P | M186V | E503D | G47N |
| Change | | | | | | |
| Type of | Missense | Missense | Missense | Missense | Missense | Missense |
| Mutation | | | | | | |

Table Legend: n/a=not applicable; F/u=follow up; Tx=heart-lung transplant; N=normal; PAPm=mean pulmonary artery pressure; RAPm=mean right atrial pressure; CI=cardiac index; PVR=pulmonary vascular resistance; SVR=systemic vascular resistance; MVSaO2=mixed venous oxygen saturation; SaO2=systemic oxygen saturation; C=Type C complete AV canal defect.

The six new missense BMPR2 mutations were in exons 2, 3, 5 and 11. Mutations in exons 2 and 3 are in the extracellular domains of BMPR2 (hence might interfere with heterodimer formation or ligand binding), exon 5 are in the kinase domain (responsible for phosphorylation) and exon 11 are in the long cytoplasmic tail. Four of the changes in predicted protein sequence caused by the mutations (adults #1, #2 and children #4, #5) alter amino acids that are conserved in evolution across human, mouse, chicken, frog and pufferfish. In contrast, the altered amino acid is conserved only in man and mouse in patients #3 and #6 (data not shown).

The 319T>C mutation in exon 3 and the 140G>A mutation in exon 2 were spontaneous as these two mutations were not found in either parent of the BMPR2-positive PAH adult with an atrioventricular canal (#3) or the child with the rare aortopulmonary window and ventricular septal defect (#6). DNAs were not available from the parents of the other mutation-positive patients.

Discussion

This is the first report of BMPR2 mutations in adults and children with PAH/CHD in whom the PAH is due to pulmonary vascular obstructive disease. The 6% frequency in a combined cohort of 40 adults and 66 children is similar to the 8% frequency of BMPR2 mutations reported for PAH with fenfluramine derivatives and in contrast to a 26% frequency in IPAH and an approximately 50% frequency in familial PAH. A recent NHLBI/ORD workshop suggests a 5-10% frequency for IPAH as do our unpublished observations.

BMPR2 mutations were found in three adults with atrioventricular canal type C and in three children with an atrial septal defect and patent ductus arteriosus, an atrial septal defect/patent ductus arteriosus and partial anomalous pulmonary venous return and a rare conotruncal CHD. None of the atrial septal defects were primum. It is suspected that as larger numbers of patients with PAH/CHD are studied, BMPR2 mutations may be found in more types of CHD. In fact, the methodology used here would miss mutations leading to large deletions and mutations affecting the promoter region of BMPR2. The failure to find BMPR2 mutations in the children with atrioventricular canal type C defects may be due to the small sample size/and or the association with Down syndrome. Five of the 6 mutation-negative children and one of the mutation-positive adults had Down syndrome. Atrioventricular canal defects are one of the most frequent CHDs that occur with trisomy 21. Therefore these defects may result from a different genetic mechanism than those that occur without a recognized chromosomal genetic syndrome. The child with atrial septal defect and patent ductus arteriosus (#4) also had a ring chromosome 14, a rare abnormality associated with CHD, mental retardation and seizures. The onset of disease in individuals with PAH is thought to require a combination of two or more genetic or environmental factors, as in cancer. To speculate, the interplay between a congenital syndrome, a CHD and a BMPR2 mutation could provide the required two or more “hits.”

The six novel missense BMPR2 mutations in exons 2, 3, 5 and 11 have the potential to be deleterious by changing the protein sequence at evolutionary conserved amino acids and hence alter BMPR2 function. It is also formally possible that these DNA sequence variations could be nonpathogenetic polymorphisms. This is unlikely as they have not been reported in the literature, nor observed in more than 196 healthy individuals and over a 1000 other PAH chromosomes screened here. Four of the six missense mutations were at sites conserved in evolution from man to the pufferfish whereas the other two sites were conserved only in man and mouse. Spontaneous BMPR2 mutations have also been reported previously in familial PAH and in sporadic IPAH. Unfortunately, parental DNAs were not available (for all patients) to determine if spontaneous mutations are a universal finding in this category of PAH/CHD.

The types of CHD found in the BMPR2-positive patients are in accordance with reports regarding the role of the BMP pathway in embryonic cardiac development. Homozygous BMPR2 knock-out mice die at gastrulation whereas no abnormalities have been reported for the heterozygous mouse. Recently, Jiao and coworkers using a Cre/loxp recombination conditional knockout demonstrated that BMP-4 contributes in a dose-related fashion to normal atrioventricular septation and endocardial cushion formation. In the presence of low or no BMP-4, mice had atrioventricular canals and outflow tract abnormalities. Delot and colleagues have created a mouse with a truncated extracellular domain of BMPR2, documenting embryonic lethality at E12, and absence of the septation of the outflow tract and aortic arch interruption, the anatomic correlate of persistant truncus arteriosus type 4A in humans. Because BMP ligands bind to a heterodimer comprised of BMPR2 with BMPR1a and/or BMPR1b to initiate signaling, mutations of either a ligand or of BMPR2 could be predicted to interfere with signaling.

This study was initiated to provide a preliminary catalogue of BMPR2 mutations in patients with PAH/CHD in whom the PAH was due to pulmonary vascular obstructive disease. As BMPR2 mutations have not been previously investigated in either children or adults with CHD without PAH, it is presently impossible to differentiate the role of increased flow versus genetic mutations in predisposing to pulmonary vascular disease. Although the definition of IPAH requires the exclusion of other causes, one may find small anatomic congenital systemic-to-pulmonary shunts. Whether these represent unrelated phenomena or genetic predisposition for IPAH with a hemodynamically insignificant congenital systemic-to-pulmonary shunt triggering the onset of the pulmonary vascular disease remains uncertain. Although pulmonary vascular disease associated with congenital systemic-to-pulmonary shunts usually follows a period of increased pulmonary blood flow, it may occur in patients who never manifested a large left to right shunt. Support for this comes from the observation of severe progressive PAH following repair of atrial septal defects in two children whose mothers died from IPAH, but by definition classifiable as familial PAH. Extrapolation from the presence of BMPR2 mutations in familial PAH and sporadic IPAH suggests BMPR2 mutations may be a risk factor for PAH/CHD. It is anticipated that further functional investigations of specific members of the human BMP/TGF-B pathway, aided by conditional murine knock-outs, will increase our knowledge of the cause(s) and interrelationship(s) between various CHDs, PAH, and the development of pulmonary vascular obstructive disease.

### List of References

1. Abenhaim L, Moride Y, Brenot F, Rich S, Benichou J, Kurz X, Higenbottam T, Oakley C, Wouters E, Aubier M, Simonneau G, Begaud B (1996) Appetite-suppressant drugs and the risk of primary pulmonary hypertension. International Primary Pulmonary Hypertension Study Group. N Engl J Med 335 (9): 609-616.
2. Altschul S F, Gish W, Miller W, Myers E W, Lipman D J (1990) Basic local alignment search tool. J Mol Biol 215 (3):403-410.
3. Barst R J, Rubin L J, Long W A, McGoon M D, Rich S, Badesch D B, Groves B M, Tapson V F, Bourge R C, Brundage B H (1996) A comparison of continuous intravenous epoprostenol (prostacyclin) with conventional therapy for primary pulmonary hypertension. The Primary Pulmonary Hypertension Study Group. N Engl J Med 334 (5):296-302.
4. Beppu H. Kawabata M, Hamamoto T, Chytil A, Minowa O, Noda T, Miyazono K (2000) BMP type II receptor is required for gastrulation and early development of mouse embryos. Dev Biol 221 (1):249-258.
5. Beppu H, Minowa 0, Miyazono K, Kawabata M (1997) cDNA cloning and genomic organization of the mouse BMP type II receptor. Biochem Biophys Res Commun 235 (3):499-504.
6. Clayton D. (1999) A Generalization of the Transmission/Disequilibrium Test for Uncertain-Haplotype Transmission. Am J Hum Genet 65 (4):1170-1177.
7. Cottingham R W, Jr., Idury R M, Schaffer A A (1993) Faster sequential genetic linkage computations. Am J Hum Genet 53:252-263.
8. D'Alonzo G E, Barst R J, Ayres S M, Bergofsky E H, Brundage B H, Detre K M, Fishman A P, Goldring R M, Groves B M, Kernis J T (1991) Survival in patients with primary pulmonary hypertension. Results from a national prospective registry. Ann Intern Med 115 (5):343-349.
9. Deng Z, Haghighi F, Helleby L, Vanterpool K, Horn E M, Barst R J, Hodge S E, Morse J H, Knowles J A (2000) Fine mapping of PPH1, a gene for familial primary pulmonary hypertension, to a 3-cM region on chromosome 2q33. Am J Respir Crit Care Med 161 (3 Pt 1):1055-1059.
10. Douglas J G, Munro J F, Kitchin A H, Muir A L, Proudfoot A T (1981) Pulmonary hypertension and fenfluramine. Br Med J (Clin Res Ed) 283 (6296):881-883.
11. Kawabata M, Chytil A, Moses H L (1995) Cloning of a novel type II serine/threonine kinase receptor through interaction with the type I transforming growth factor-beta receptor. J Biol Chem 270 (10):5625-5630.
12. Kimura N, Matsuo R, Shibuya H, Nakashima K, Taga T (2000) BMP2-induced Apoptosis Is Mediated by Activation of the TAK1-p38 Kinase Pathway That Is Negatively Regulated by Smad6. J Biol Chem 275 (23):17647-17652.
13. Kruglyak L, Daly M J, Reeve-Daly M P, Lander E S (1996) Parametric and nonparametric linkage analysis: a unified multipoint approach. Am J Hum Genet 58 (6):1347-1363.
14. Lee S D, Shroyer K R, Markham N E, Cool C D, Voelkel N F, Tuder R M (1998) Monoclonal endothelial cell proliferation is present in primary but not secondary pulmonary hypertension. J Clin Invest 101 (5):927-934.
15. Liu F, Ventura F, Doody J, Massague J (1995) Human type II receptor for bone morphogenic proteins (BMPs): extension of the two-kinase receptor model to the BMPs. Mol Cell Biol 15 (7):3479-3486.
16. Loyd J E, Butler M G, Foroud T M, Conneally P M, Phillips J A, Newman J H (1995) Genetic anticipation and abnormal gender ratio at birth in familial primary pulmonary hypertension. Am J Respir Crit Care Med 152 (1):93-97.
17. Massague J (1998) TGF-beta signal transduction. Annu Rev Biochem 67:753-791.
18. Morse J H and Barst R J (1994) Immunological disturbances in primary pulmonary hypertension. Sem Resp Crit Care 15:222-229.
19. Morse J. H., Jones, A., DiBenedetto, A., Hodge, S. E., and Nygaard, T. G. (1996) Genetic mapping of primary pulmonary hypertension: Evidence for linkage to chromosome 2 in a large family. Circulation 94 (8):I-46.
20. Morse J H, Jones A C, Barst R J, Hodge S E, Wilhelmsen K C, Nygaard T G (1997) Mapping of familial primary pulmonary hypertension locus (PPH1) to chromosome 2q31-q32. Circulation 95 (12):2603-2606.
21. Nichols W C, Koller D L, Slovis B, Foroud T, Terry V H, Arnold N D, Siemieniak D R, Wheeler L, Phillips J A, Newman J H, Conneally P M, Ginsburg D, Loyd J E (1997) Localization of the gene for familial primary pulmonary hypertension to chromosome 2q31-32. Nat Genet 15 (3):277-280.
22. Nohno T, Ishikawa T., Saito T, Hosokawa K, Noji S, Wolsing D H, Rosenbaum J S (1995.) Identification of a human type II receptor for bone morphogenetic protein-4 that forms differential heteromeric complexes with bone morphogenetic protein type I receptors. J Biol Chem 270 (38):22522-22526.
23. O'Donovan M C, Oefner P J, Roberts S C, Austin J, Hoogendoorn B, Guy C, Speight G, Upadhyaya M, Sommer S S, McGuffin P (1998) Blind Analysis of Denaturing High-Performance Liquid Chromatography as a Tool for Mutation Detection. Genomics 52 (1):44-49.
24. Pasque M K, Trulock E P, Cooper J D, Triantafillou A N, Huddleston C B, Rosenbloom M, Sundaresan S, Cox J L, Patterson G A (1995) Single lung transplantation for pulmonary hypertension. Single institution experience in 34 patients. Circulation 92 (8):2252-2258.
25. Rich S, Dantzker D R, Ayres S M, Bergofsky E H, Brundage B H, Detre K M, Fishman A P, Goldring R M, Groves B M, Koerner S K (1987) Primary pulmonary hypertension. A national prospective study. Ann Intern Med 107 (2):216-223.
26. Rosenzweig B L, Imamura T, Okadome T, Cox G N, Yamashita H, ten Dijke P, Heldin C H, Miyazono K (1995) Cloning and characterization of a human type II receptor for bone morphogenetic proteins. Proc Natl Acad Sci USA 92 (17):7632-7636.
27. Soda H, Raymond E, Sharma S, Lawrence R, Cerna C, Gomez L, Timony G A, Von Hoff D D, Izbicka E (1998) Antiproliferative effects of recombinant human bone morphogenetic protein-2 on human tumor colony-forming units. Anticancer Drugs 9 (4):327-331.
28. Topper J N, Cai J, Qiu Y, Anderson K R, Xu Y Y, Deeds J D, Feeley R, Gimeno C J, Woolf E A, Tayber O, Mays G G, Sampson B A, Schoen F J, Gimbrone M A, Jr., Falb D (1997) Vascular MADS: two novel MAD-related genes selectively inducible by flow in human vascular endothelium. Proc Natl Acad Sci USA 94 (17):9314-9319.
29. Underhill P A, Jin L, Lin A A, Mehdi S Q, Jenkins T, Vollrath D, Davis R W, Cavalli-Sforza L L, Oefner P J (1997) Detection of numerous Y chromosome biallelic polymorphisms by denaturing high-performance liquid chromatography. Genome Res 7 (10):996-1005.
30. Warburton D, Schwarz M, Tefft D, Flores-Delgado G, Anderson K D, Cardoso W V (2000) The molecular basis of lung morphogenesis. Mech Dev 92 (1):55-81.
31. Wieser R, Attisano L, Wrana J L, Massague J (1993) Signaling activity of transforming growth factor beta type II receptors lacking specific domains in the cytoplasmic region. Mol Cell Biol 13 (12):7239-7247.
32. Galloway, S. M., McNatty, K. P., Cambridge, L. M., Laitinen, M. P., Juengel, J. L., Jokiranta, T. S., McLaren, R. J., Luiro, K., Dodds, K. G., Montgomery, G. W., Beattie, A. E., Davis, G. H., and Ritvos, O. (2000). Mutations in an oocyte-derived growth factor gene (BMP15) cause increased ovulation rate and infertility in a dosage-sensitive manner [In Process Citation]. Nat. Genet. 25, 279-283.
33. Morse, J. H. and Barst, R. J. (1997). Detection of familial primary pulmonary hypertension by genetic testing [letter]. N. Engl. J. Med. 337, 202-203.

What is claimed:

1. A method of detecting whether a subject is predisposed to, or afflicted with, pulmonary arterial hypertension (PAH) which comprises (A) obtaining a suitable sample comprising a nucleic acid encoding bone morphogenetic protein receptor II from the subject; and (B) detecting in the nucleic acid encoding bone morphogenetic protein receptor II whether a mutation is present which is not present in a nucleic acid encoding wildtype bone morphogenetic protein receptor-II,
wherein the mutation described relative to a difference from the sequence encoding wildtype bone morphogenetic protein receptor II set forth in SEQ ID NO:1 is selected from the group consisting of:
(1) a substitution of an adenosine nucleotide located at position 125 with a guanosine nucleotide;
(2) a substitution of a guanosine nucleotide located at position 140 with an adenosine nucleotide;
(3) a substitution of an adenosine nucleotide located at position 304 with a guanosine nucleotide;
(4) a substitution of a thymidine nucleotide located at position 319 with a cytosine nucleotide;
(5) a substitution of an adenosine nucleotide located at position 556 with a guanosine nucleotide;
(6) a substitution of an adenosine nucleotide located at position 1509 with a cytosine nucleotide;
wherein the presence of such a mutation indicates that the subject is predisposed, to or afflicted with, pulmonary arterial hypertension (PAH).

2. The method of claim 1, wherein the subject is human.

3. The method of claim 1, wherein the subject has congenital heart disease.

4. A method of detecting whether a subject is predisposed to, or afflicted with, pulmonary arterial hypertension (PAH) which comprises (A) obtaining a suitable sample comprising bone morphogenetic protein receptor II from the subject; and (B) detecting in the bone morphogenetic protein receptor II whether a mutation is present which is not present in wildtype bone morphogenetic protein receptor-II,
wherein the mutation described relative to a difference from the wildtype bone morphogenetic protein receptor II sequence set forth in SEQ ID NO:2 is selected from the group consisting of:
(1) a substitution of a glutamine residue located at position 42 with an arginine residue;
(2) a substitution of a glycine residue located at position 47 with an asparagines residue;
(3) a substitution of a threonine residue located at position 102 with an alanine residue;
(4) a substitution of a serine residue located at position 107 with a proline residue;
(5) a substitution of a methionine residue located at position 186 with a valine residue;
(6) a substitution of a glutamic acid residue located at position 503 with an aspartic acid residue;
wherein the presence of such a mutation indicates that the subject is predisposed, to or afflicted with, pulmonary arterial hypertension (PAH).

5. The method of claim 4, wherein the subject is human.

6. The method of claim 4, wherein the subject has congenital heart disease.
